(19) United States
(12) Patent Application Publication
(10) Pub. No.: US 2025/0035649 A1
(43) Pub. Date: Jan. 30, 2025

(54) System for Detection, Monitoring, and Treatment of Kidney Disease

(71) Applicant: Innovo Bio, Boston, MA (US)

(72) Inventors: Nathan N. Lee, Boston, MA (US); Joseph V. Bonventre, Boston, MA (US)

(21) Appl. No.: 18/783,463

(22) Filed: Jul. 25, 2024

Related U.S. Application Data
(60) Provisional application No. 63/515,598, filed on Jul. 26, 2023.

(51) Int. Cl. G01N 33/68

(52) U.S. Cl. CPC G01N 33/6893; G01N 2800/347

(57) ABSTRACT

Provided herein are approaches, including but not limited to biomarkers, methods, assays, and kits for early detection and diagnosis and monitoring of kidney injury and chronic kidney disease (CKD) along with treatment options to prevent or mitigate the progression of kidney injury and CKD. The present disclosure utilizes the fact that specific biomarkers are present in biological samples, such as urine or serum, at altered levels in subjects with presence or risk of kidney injury or CKD in different stages as compared with subjects that have no kidney injury or CKD and applies this to inventive and important diagnostic and therapeutic technologies.

### FIELD OF THE INVENTION

The present invention relates to chronic kidney disease and methods for its diagnosis, prevention, and treatment in animals (including, but not limited to cats and dogs) and human.

### BACKGROUND OF THE INVENTION

Kidney disease describes a class of conditions in which the kidney is damaged and/or has varying degrees of defects in its functions such as filtration and removal of waste products from the blood, depending on its type and severity.

Chronic kidney disease (CKD) is one of the biggest and fastest growing health concerns around the world for both animals and humans. In humans, CKD can lead to dialysis and other conditions such as heart disease with associated medical costs totaling in the billions of dollars.

CKD can develop as a result of many different factors, such as genetic predisposition, diabetes, and acute kidney injury. The degree of kidney injury is also associated with an incremental increase in long-term mortality. Moreover, currently used markers of kidney function/injury, such as serum creatinine levels, are poor at detecting the early stage of the disease or discriminating long-term outcome of kidney disease. Regardless of the initiating factor, CKD results in a high proportion of patients that require long-term dialysis and renal replacement therapy. These treatments are expensive, time consuming, and can result in severe side effects, while their accessibility is also limited.

Thus, development of a biomarker that allows prediction and early identification of CKD in patients is a clinical tool having great need in the art.

The traditional blood and urine biomarkers for CKD such as creatinine, blood urea nitrogen, SDMA, GFR that have been used in diagnosis and prognosis of CKD are not sufficiently sensitive and do not adequately reflect actual damage in the kidney. By the time the currently existing biomarkers are abnormal, the kidneys have already lost more than half of their function and the disease is not reversible. The lack of effective biomarkers for kidney disease has led to the lack of effective therapies. Thus, development of effective biomarkers for kidney disease is critical for early detection, prevention, management, monitoring, and treatment.

The rationale for screening for early-stage CKD includes the high and rising prevalence of CKD, its known risk factors, severity of its health consequences, costly and ineffective treatment options for advanced CKD, its long asymptomatic phase, and the availability of treatments for early-stage CKD that may alter the course of CKD and reduce its progression and complications or associated health conditions.

### SUMMARY OF THE INVENTION

Provided herein are biomarkers, methods, assays, and kits for early detection and diagnosis and monitoring of kidney disease along with treatment options to prevent or mitigate the progression of CKD. The present invention is based on the discovery that specific biomarkers are present in urine or serum at altered levels in subjects with kidney injury or CKD in different stages as compared with subjects that have no kidney injury or CKD. Accordingly, the invention is directed to methods for diagnosis of kidney injury or CKD by determining and monitoring the levels of at least one biomarker protein in a biological sample, such as urine and serum as well as devices, components, and system to measure said biomarkers. Biomarkers present at altered levels in urine or serum include the proteins listed in, for example, Tables 1, 2, and 3.

Accordingly, one aspect of the invention provides at least one biomarker specific for the diagnosis and monitoring of kidney injury or CKD in a subject in need thereof.

One embodiment of this aspect, and all aspects described herein, provides a biomarker for kidney injury or CKD, the biomarkers in, for example, Tables 1, 2, and 3, that is significantly increased/reduced in patients with kidney injury or CKD. The kidney injury or CKD may be any degree or stage either prior to or after the development of a formal diagnosis of CKD in a patient. The sample may be urine, blood, plasma, serum, or saliva, preferably the sample may be urine or serum.

Another embodiment of this aspect, and all aspects described herein, provides a panel of biomarkers for kidney injury or CKD, each of which is differentially altered in kidney injury or CKD patients, and provides comparative value in the diagnosis and prognosis of kidney injury or CKD. In one embodiment of this aspect, and all aspects described herein, the kidney injury/CKD biomarker panel comprises any combination of biomarkers in, for example, Table 1.

In a further embodiment, biomarkers may be selected from the Table 2 or Table 3 following more limited listing.

In all such embodiments of this aspect and all aspects described herein, an agent specific for total protein or a normalizing protein or factor, such as albumin, creatinine or SDMA, may also be included, or an assay to measure the level or concentration of total protein or a normalizing protein or factor may be performed in order to provide a level or concentration to which the panel of biomarkers can be normalized to, in order to permit various comparisons, for example, between subject samples, or between a series of samples isolated from one subject at different timepoints.

In one embodiment of the invention, kidney injury/CKD biomarker (those in Tables 1, 2, and 3) levels present in a biological sample, such as urine or serum, are measured by contacting the test sample, or preparation thereof, with an agent, such as an antibody-based or aptamer-based agent, that specifically binds to at least one kidney injury/CKD biomarker, or to a portion thereof such as those biomarkers listed in Tables 1, 2, and 3. The agent forms a complex with the one or more biomarkers desired to be tested, which can be used in assays to determine the biomarker concentration or level. Any means known to those skilled in art can be used to assess biomarker levels. For example, biomarker levels can be assessed by ELISA, multiplex bead assay, or mass spectrometry, including SELDI mass spectrometry and other technologies.

Another embodiment of this aspect, and all aspects described herein, provides a method for determining the treatment for kidney injury or CKD patients and monitoring progression or efficacy of a treatment for kidney injury or CKD patients, which include measuring the amount of selected panel of biomarkers in a urine or serum sample obtained from a subject with kidney injury or CKD over time, which may include after administering of a treatment or treatment regimen, or for monitoring over time, or both. Wherein the biomarkers are at least one or more of the proteins listed in Tables 1, 2, and 3, to determine progression of kidney injury or CKD or efficacy of a treatment.

Accordingly, in one embodiment of this aspect, the method comprises (a) measuring a level or concentration of at least one biomarker in a panel of biomarkers comprising those listed in Tables 1, 2, and 3; and (b) comparing the level or concentration of the at least one biomarker with a reference level or concentration of the at least one biomarker, wherein an increase or reduction in the level or concentration of at least one biomarker in a panel of biomarkers comprising those listed in Tables 1, 2, and 3 in the sample relative to the reference level or concentration of said at least one biomarker indicates a need to administer to the subject a specific prevention, management or treatment plan for CKD. In some embodiments, the biological sample is a urine or serum or plasma or saliva sample.

Another embodiment of this aspect, and all aspects described herein, provides a method for determining the plans for prevention and management of CKD.

In another aspect, the invention provides for use of kits that comprise means for measuring at least one kidney injury/CKD biomarker from the listed proteins in Tables 1, 2, and 3 in a biological sample to screen for kidney injury or CKD in a method according to any of the proceeding aspects of the invention. The kit may comprise a container for holding a biological sample (e.g. urine or serum sample), control sample or reagent to show negative or positive control, and at least one agent, such as an antibody or aptamer, that specifically binds at least one kidney injury/CKD biomarker noted herein for use in determining the level, concentration or the presence of at least one kidney injury/CKD biomarker in a biological sample, such as a urine or serum sample. The at least one agent, such as an antibody or aptamer, may be present as a part of the kit in a container to receive biological sample, within a dipstick to expose to biological sample, in a separate container to receive a smaller quantity of the biological sample or to be later administered in the biological sample, disposed on a strip, cartridge, assay, or the like.

Another aspect described herein relates to use of computer software with specific algorithms or artificial intelligence to analyze test results of the kidney injury/CKD biomarker panel for predicting and diagnosing kidney injury or CKD of at least one individual by taking different variables, such as patient medical records and history, kidney injury/CKD biomarker panel test results, normalization, and progression, into account.

In one embodiment of the invention, the invention may be used for prevention or treatment of CKD patients at all different stages. At least one kidney injury/CKD biomarkers in Tables 1, 2, and 3 can be a target of therapeutic treatment (e.g. antibody or drug) in kidney injury or CKD patient. At least one kidney injury/CKD biomarker in Tables 1, 2, and 3 can be used as a therapeutic agent (e.g. therapeutic peptide/protein).

### BRIEF DESCRIPTION OF THE DRAWINGS

FIG. 1 provides a flow chart of one embodiment of the present disclosure.

### TABLES

TABLE 1
List of kidney injury/CKD biomarkers.

| | Change |
|---|---|
| Biomarker Name | in CKD |
| Apolipoprotein A-I (APOA1) | Increased |
| Beta-2-glycoprotein 1 (APOH) | Increased |
| cAMP responsive element binding protein 3 like 3 | Reduced |
| (CREB3L3) | |
| CD44 antigen | Reduced |
| Clusterin (CLU) | Increased |
| Collagen type I alpha 1 chain (COL1A1) | Reduced |
| Collagen type I alpha 2 chain (COL1A2) | Reduced |
| Collagen type VI alpha 3 chain (COL6A3) | Reduced |
| Complement factor B (CFB) | Increased |
| Deoxyribonuclease (DNASE1) | Reduced |
| Family with sequence similarity 174 member A (FAM174A) | Reduced |
| Fetuin B (FETUB) | Increased |
| GC vitamin D binding protein (VDBP) | Increased |
| Gelsolin | Increased |
| Hemopexin (HPX) | Increased |
| Insulin like growth factor binding protein 7 (IGFBP7) | Reduced |
| Kallikrein 1 (KLK1) | Reduced |
| Kininogen 1 (KNG1) | Reduced |
| Lipase A, lysosomal acid type (LIPA) | Reduced |
| Lymphocyte antigen 6 family member D (LY6D) | Reduced |
| Napsin A aspartic peptidase (NAPSA) | Reduced |
| Nuclear transport factor 2 (NUTF2) | Reduced |
| Phosphoinositide-3-kinase interacting protein 1 (PIK3IP1) | Reduced |
| Plasminogen | Increased |
| Polymeric immunoglobulin receptor (PIGR) | Reduced |
| Protein AMBP | Increased |
| Transferrin | Increased |
| Urokinase-type plasminogen activator (PLAU) | Reduced |
| Uromodulin | Reduced |
| Uteroglobin (ASRGL1) | Reduced |
| WAP four-disulfide core domain protein 2 (WFDC2) | Reduced |

TABLE 2
List of kidney injury/CKD biomarkers.

| | Change |
|---|---|
| Description | in CKD |
| Apolipoprotein A-I (APOA1) | Increased |
| cAMP responsive element binding protein 3 like 3 | Reduced |
| (CREB3L3) | |
| CD44 antigen | Reduced |
| Collagen type I alpha 1 chain (COL1A1) | Reduced |
| Collagen type I alpha 2 chain (COL1A2) | Reduced |
| Collagen type VI alpha 3 chain (COL6A3) | Reduced |
| Complement factor B (CFB) | Increased |
| Deoxyribonuclease (DNASE1) | Reduced |
| Family with sequence similarity 174 member A (FAM174A) | Reduced |
| Gelsolin | Increased |
| Kallikrein 1 (KLK1) | Reduced |
| Kininogen 1 (KNG1) | Reduced |
| Lipase A, lysosomal acid type (LIPA) | Reduced |
| Lymphocyte antigen 6 family member D (LY6D) | Reduced |
| Napsin A aspartic peptidase (NAPSA) | Reduced |
| Nuclear transport factor 2 (NUTF2) | Reduced |
| Phosphoinositide-3-kinase interacting protein 1 (PIK3IP1) | Reduced |
| Plasminogen | Increased |
| Polymeric immunoglobulin receptor (PIGR) | Reduced |
| Protein AMBP | Increased |
| Transferrin | Increased |
| Uteroglobin (ASRGL1) | Reduced |
| WAP four-disulfide core domain protein 2 (WFDC2) | Reduced |

TABLE 3
List of kidney injury/CKD biomarkers.

| | Change |
|---|---|
| Description | in CKD |
| Apolipoprotein A-I (APOA1) | Increased |
| cAMP responsive element binding protein 3 like 3 | Reduced |
| (CREB3L3) | |
| CD44 antigen | Reduced |
| Collagen type I alpha 1 chain (COL1A1) | Reduced |
| Collagen type I alpha 2 chain (COL1A2) | Reduced |
| Collagen type VI alpha 3 chain (COL6A3) | Reduced |
| Complement factor B (CFB) | Increased |
| Deoxyribonuclease (DNASE1) | Reduced |
| Family with sequence similarity 174 member A (FAM174A) | Reduced |
| Gelsolin | Increased |
| Kallikrein 1 (KLK1) | Reduced |
| Kininogen 1 (KNG1) | Reduced |
| Lipase A, lysosomal acid type (LIPA) | Reduced |
| Lymphocyte antigen 6 family member D (LY6D) | Reduced |
| Napsin A aspartic peptidase (NAPSA) | Reduced |
| Nuclear transport factor 2 (NUTF2) | Reduced |
| Phosphoinositide-3-kinase interacting protein 1 (PIK3IP1) | Reduced |
| Plasminogen | Increased |
| Polymeric immunoglobulin receptor (PIGR) | Reduced |
| Transferrin | Increased |

TABLE 4
List of biomarkers increased in CKD.

| Biomarker Name | Fold Increased |
|---|---|
| Apolipoprotein A-I (APOA1) | 14.60 |
| Beta-2-glycoprotein 1 (APOH) | 4.42 |
| Clusterin (CLU) | 2.80 |
| Complement factor B (CFB) | 5.69 |
| Fetuin B (FETUB) | 2.84 |
| GC vitamin D binding protein (VDBP) | 3.38 |
| Gelsolin | 3.42 |
| Hemopexin (HPX) | 7.61 |
| Plasminogen | 4.49 |
| Protein AMBP | 4.57 |
| Transferrin | 2.91 |

TABLE 5
List of biomarkers decreased in CKD.

| | Fold |
|---|---|
| Biomarker Name | Reduced |
| cAMP responsive element binding protein 3 like 3 | 0.13 |
| (CREB3L3) | |
| CD44 antigen | 0.05 |
| Collagen type I alpha 1 chain (COL1A1) | 0.43 |
| Collagen type I alpha 2 chain (COL1A2) | 0.32 |
| Collagen type VI alpha 3 chain (COL6A3) | 0.06 |
| Deoxyribonuclease (DNASE1) | 0.03 |
| Family with sequence similarity 174 member A (FAM174A) | 0.26 |
| Insulin like growth factor binding protein 7 (IGFBP7) | 0.06 |
| Kallikrein 1 (KLK1) | 0.03 |
| Kininogen 1 (KNG1) | 0.33 |
| Lipase A, lysosomal acid type (LIPA) | 0.29 |
| Lymphocyte antigen 6 family member D (LY6D) | 0.03 |
| Napsin A aspartic peptidase (NAPSA) | 0.23 |
| Nuclear transport factor 2 (NUTF2) | 0.14 |
| Phosphoinositide-3-kinase interacting protein 1 (PIK3IP1) | 0.04 |
| Polymeric immunoglobulin receptor (PIGR) | 0.28 |
| Urokinase-type plasminogen activator (PLAU) | 0.20 |
| Uromodulin | 0.03 |
| Uteroglobin (ASRGL1) | 0.06 |
| WAP four-disulfide core domain protein 2 (WFDC2) | 0.11 |

### DETAILED DESCRIPTION OF THE INVENTION

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

Chronic kidney disease (CKD) is associated with high morbidity and mortality. The lack of sensitive and specific biomarkers to predict or detect CKD at an early stage has greatly impeded the development of therapeutic strategies to improve outcomes of CKD. The diagnostic approach to CKD at the moment is able to detect CKD only after kidney function is severely compromised, which is already too late for treatment or prevention of progression in most cases. Kidney injury could lead to development of CKD or advanced progression of CKD.

The embodiments of the present invention provide for the diagnosis capability and therapeutic options for kidney injury or CKD utilizing a panel of kidney injury/CKD biomarkers in Tables 1, 2, and 3. These biomarkers were analyzed in a cross-sectional comparison of patients with or without CKD.

Protein level of biomarkers in patients with CKD was significantly altered, either higher or lower, compared to those without CKD. Mass-spec analysis was used to identify the panel of biomarker proteins with altered levels in CKD and non-CKD patients.

Several of the biomarkers provided for herein in the context of kidney injury or CKD have been characterized previously to some extent.

The present inventors have found that detection of altered levels of a biomarker selected from the group consisting of kidney injury/CKD biomarkers in, for example, Tables 1, 2, and 3 in urine or serum samples can be used to predict or identify patients suffering from kidney injury or CKD in different stages of the kidney injury or CKD progression. Thus, the present invention may be used to predict development of CKD or diagnose any stage of kidney injury or CKD, including the early-stage CKD. In the context of the present invention, “early-stage CKD” is understood to mean any of the first or second stages of CKD as defined by the standard classifications.

In a first aspect, the present invention provides a method of identifying patients with risk of CKD or suffering from kidney injury or CKD or staging the progression of CKD in a patient comprising measuring the amount of at least one biomarker in a sample obtained from the subject, and determining whether the amount of the biomarker is altered compared to a normal control, wherein the biomarkers used are selected from the group consisting of kidney injury/CKD biomarkers in Tables 1, 2, and 3. Tables 1, 2, and 3 presents the list of kidney injury/CKD biomarkers increased or decreased in CKD patients compared to control patients. An increased expression level (when compared to a healthy non-diseased control) of biomarkers in Tables 1, 2, 3, and 4 indicates the presence of or risk of CKD. A reduced expression level (when compared to a healthy non-diseased control) of biomarkers in Tables 1, 2, 3, and 5 indicates the presence of or risk of kidney injury or CKD.

Where one or more biomarkers are used in the invention, a suitable mathematical, machine learning, or artificial intelligence algorithms can be utilized to analyze the results and determine the status of kidney injury or CKD for the subject. Patient medical records and history will be included in the analysis.

The present inventors have further found that detection of altered levels of combinations of two or more biomarkers selected from the group consisting of proteins in Tables 1, 2, and 3 can be used to identify patients with risk of CKD or suffering from kidney injury or CKD or to stage the progression of the disease.

The present disclosure provides a method to support the decision to implement therapeutic intervention in a patient suspected of having renal disease comprising measuring the amount of a biomarker in a sample obtained from the subject, and determining whether the amount of the biomarker is altered compared to a normal control, wherein the biomarker is selected from the group consisting of kidney injury/CKD biomarkers in Tables 1, 2, and 3.

The present disclosure also provides therapeutic options for prevention or treatment of CKD patients. The present inventors have found kidney injury/CKD biomarker proteins that are increased or reduced in urine or serum samples of CKD patients. Thus, at least one kidney injury/CKD biomarker proteins in Tables 1, 2, and 3 that are increased in kidney injury or CKD patients can be used as target of therapeutic treatment (e.g. antibody or drug) while at least one kidney injury/CKD biomarker proteins in Tables 1, 2, and 3 that are decreased in kidney injury or CKD patients can be used as therapeutic agent (e.g. therapeutic peptide/protein).

### Determining the Levels and Concentrations of Kidney Injury/CKD Biomarkers

In one aspect, the invention provides a method for diagnosing chronic kidney disease (CKD) in a subject. In one embodiment, the method comprises measuring the concentration of a normalizing protein or factor, such as albumin or creatinine, and at least one biomarker in a biological sample obtained from a subject; and comparing the concentration of the at least one biomarker to the concentration of normalizing protein or factor in the sample to determine whether the subject has kidney injury or CKD. In one embodiment, the biological sample is a urine or serum sample. In one embodiment, the at least one biomarker is selected from a panel of biomarkers in Tables 1, 2, and 3. In all such embodiments, an altered level or concentration of at least one biomarker indicates the presence of or risk of kidney injury or CKD.

In another embodiment, the method and system comprises contacting a collected sample obtained from a subject in need thereof with at least one agent specific for at least one biomarker selected from a panel of biomarkers in Tables 1, 2, and 3 and at least one agent specific for a normalizing protein or factor, such as albumin or creatinine, where the agents specific for at least one biomarker and the normalizing protein or factor are used in an assay to determine the level or concentration of the at least one biomarker and the level or concentration of the normalizing protein or factor; and diagnosing a subject with kidney injury or CKD based on the level or concentration of the at least one biomarker present in the sample. In some embodiments, the method further comprises determining a therapeutic treatment for the subject. In one embodiment, the at least one biomarker is selected from a panel of biomarkers in Tables 1, 2, and 3. In one embodiment, the concentration of the at least one biomarker is compared with the level or concentration of the normalizing protein or factor, where an altered level or concentration of at least one biomarker indicates the presence of or risk of kidney injury or CKD. In other embodiments, the level or concentration of the biomarker protein is measured by measuring the activity of the biomarker.

In one aspect, the method comprises determining the increase or decrease of at least two kidney injury/CKD biomarkers (e.g., biomarkers in Tables 1, 2, and 3 or total protein) in a biological sample (e.g., a urine or serum sample) obtained from a patient, wherein the increase or decrease of at least one marker indicates the presence of or risk of kidney injury or CKD.

In another embodiment, the methods involve determining the levels or concentrations of at least one kidney injury/CKD biomarker (e.g., biomarkers in Tables 1, 2, and 3) in a test sample obtained from a patient being tested for kidney injury or CKD and comparing the observed levels with the levels of the biomarker found in a control sample (healthy individual without kidney injury or CKD). Altered levels of at least one biomarker in the patient sample compared to the normal control indicates the presence of or risk of kidney injury or CKD. The levels of biomarkers can be represented by arbitrary units, for example as units obtained from a densitometer, luminometer, or an ELISA plate reader.

In one embodiment of the aspect, a secondary diagnostic step can be performed. For example, if a level of at least one kidney injury/CKD biomarker is found to indicate the presence of kidney injury or CKD, then an additional method of detecting the kidney injury or CKD can be performed to confirm the disease or further assess the extent of kidney injury or CKD. Any of a variety of additional diagnostic steps can be used, such as ultrasound, PET scanning, MRI, or any other imaging techniques, biopsy, clinical examination, ductogram, or any other method.

The present invention further provides for methods of prognostic evaluation of a patient suspected of having, or having, kidney injury or CKD. The method comprises measuring the level of at least one kidney injury/CKD biomarker in Tables 1, 2, and 3 present in a test biological sample (e.g., urine or serum) obtained from a patient and comparing the observed level with a range of at least one kidney injury/CKD biomarker level normally found in biological samples (of the same type) of healthy individuals. A high or low level for specific biomarker in Tables 1, 2, and 3 can correspond to a poor or better prognosis.

Additionally, progression of kidney injury or CKD can be assessed by following the levels or concentrations of at least one kidney injury/CKD biomarker in an individual patient. For example, changes in the patient's condition can be monitored by comparing changes in expression levels of biomarkers in Tables 1, 2, and 3 in the patient over time. Progressive change in the levels or concentrations of at least one biomarker may be indicative of increased potential for adverse outcome (e.g., mortality). Levels or concentrations of at least one kidney injury/CKD biomarker, as described herein, can be measured by any means known to those skilled in the art, including ELISA, multiplex bead, mass spectrometry, and PCR assays. The antibodies for use in the present invention can be obtained from a commercial source or prepared by well-known methods.

As used herein, the phrase “normalizing the level of the biomarker” refers to the conversion of a data value representing the level of a biomarker (e.g., biomarker in Tables 1, 2, and 3) in a sample by dividing it by the expression data value representing the level of total protein or a normalizing protein or a normalizing factor (e.g., creatinine) in the sample, thereby permitting comparison of normalized biomarker values among a plurality of samples, or to one or more reference samples or reference values.

As used herein, the term “normalizing agent”, “normalizing protein” or “normalizing factor” refers to a protein or a compound against which the amounts of a biomarker of interest are normalized to, to permit comparison of amounts of the protein of interest in different biological samples. In some embodiments, the normalizing factor is creatinine. In some embodiments, the different biological samples are from different subjects. In other embodiments, the different biological samples are from the same subject, but after different timepoints. Generally, a normalizing protein is constitutively expressed and is not differentially regulated between at least two physiological states or conditions from which samples will be analyzed, e.g., given disease and non-disease states. Thus, for example, a normalizing protein does not vary significantly in the presence and absence of e.g. kidney disease. In some embodiments, the normalizing protein is the product of a “housekeeping gene”. As referred to herein, the term “housekeeping gene” refers to a gene encoding a protein that is constitutively expressed and is necessary for basic maintenance and essential cellular functions. Some examples of normalizing proteins encoded by housekeeping genes include e.g., actin, tubulin, GAPDH, among others. In one embodiment, a housekeeping gene product is used as a normalizing protein.

The invention provides, in part, a variety of assay formats that can be used to determine the concentration or level of a biomarker or a normalizing protein or factor. Examples of assay formats include known techniques such as Western blot analysis, radioimmunoassay (hereinafter referred to as “RIA”), Immunoradiometric assay (IRMA), chemiluminescent immunoassays, such as enzyme-linked immunosorbent assay (hereinafter referred to as “ELISA”), multiplex bead assays, a fluorescence antibody method, aptamer-based method, passive hemagglutination, mass spectrometry (such as MALDI/TOF (time-of-flight), SELDI/TOF), liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, and tandem mass spectrometry HPLC.

In some embodiments, the level or concentration of each of a plurality of biomarkers can be determined simultaneously, in a multiplex fashion, by ELISA (enzyme-linked immunosorbent assay). The sample can be, for example, one of a plurality of samples obtained at one of the various timepoints from a subject in need. In some embodiments, the sample is a urine or serum sample from a subject, to be tested for determining the level or concentration of at least one biomarker according to the methods described herein. The sample (e.g., urine or serum) from the individual may further be serially diluted, according to the needs of the assay, and as known to one of ordinary skill in the art. In some embodiments, one or more of a plurality of antibodies or antigen-binding fragments specific for each of the at least one biomarker being assayed in a sample is contacted with the sample to bind any biomarker present in the sample, thus forming a complex, such as biomarker-antibody complex, biomarker-antigen-binding fragment complex, or biomarker-aptamer complex. In some embodiments, each antibody, antigen-binding fragment, or aptamer specific for a biomarker is labeled with a different label. In some embodiments, each different label is a fluorescent label. In all such embodiments, each different label has unique emission spectra, such that each antibody or aptamer can be detected individually. The levels or concentrations of each of the biomarkers can then be determined by calculating changes in the emission spectrum, wherein the relative intensity of signal from each of the fluorescent labels correlates with the number of antibodies against the particular biomarker being assayed. A series of standards having known concentrations of each of the various biomarkers being assayed permits actual quantification of the concentration of each of the biomarkers in the sample.

Other techniques can be used to detect the kidney injury/CKD biomarkers as required to practice the methods described herein, according to a practitioner's preference, and based upon the present disclosure. The suitability of a given method of distinguishing kidney injury/CKD biomarkers will depend on the ability of that method or assay to distinguish between the kidney injury/CKD biomarkers. Thus, an immunoassay can distinguish on the basis of selective binding of one and not another agent. Spectrometric approaches can be applied when a given agent will have a distinct spectrum or profile in the assay relative to others.

In other embodiments, the levels of the various kidney injury/CKD biomarkers present in a sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry.

The prognostic methods of the invention also are useful for determining a proper course of treatment for a patient having kidney injury or CKD. A course of treatment refers to the therapeutic measures taken for a patient after diagnosis or after treatment.

The present invention is also directed to commercial kits for the detection and prognostic evaluation of kidney injury or CKD. The kit can be in any configuration and is useful for performing one or more of the methods described herein for the detection of at least one kidney injury/CKD biomarker. The kits are convenient in that they supply many, if not all, of the essential reagents for conducting an assay for the detection of at least one kidney injury/CKD biomarker in a urine or serum test sample, such as described herein. In addition, the assay may be performed simultaneously with a standard or multiple standards included in the kit, such as a predetermined amount of at least one kidney injury/CKD biomarker so that the results of the test can be quantified or validated.

In one embodiment, the kit comprises components for detecting levels of at least one kidney injury/CKD biomarker in a sample of urine or serum. The kit may comprise a “dipstick” with at least one kidney injury/CKD biomarker binding agent immobilized thereon, which specifically binds a kidney injury/CKD biomarker protein. Specifically bound kidney injury/CKD biomarker can then be detected using another agent, for example, an antibody or aptamer that is detectably labeled with a calorimetric agent or radioisotope. All such components, compositions, and elements are explicitly incorporated and contemplated by this disclosure.

In other embodiments, the assay kits may contain components for competitive and non-competitive assays, radioimmunoassay (RIA), multiplex bead assays, bioluminescence and chemiluminescence assays, fluorometric assays, sandwich assays, immunoradiometric assays, dot blots, enzyme linked assays including ELISA, microtiter plates, or immunocytochemistry. For each kit the range, sensitivity, precision, reliability, specificity, and reproducibility of the assay are established by means well known to those skilled in the art.

### Methods of Optimizing Treatments for Chronic Kidney Disease

Other aspects of the invention provide methods for improving the efficacy of treatment for kidney injury or CKD, by determining the levels or concentrations of biomarkers.

Accordingly, in one embodiment of this aspect, the method comprises (a) measuring a level or concentration of at least one biomarker in a panel of biomarkers in Tables 1, 2, and 3; and (b) comparing the level or concentration of the at least one biomarker with a reference level or concentration of the at least one biomarker, wherein an increase or reduction in the level or concentration of at least one biomarker in a panel of biomarkers in Tables 1, 2, and 3 in the sample relative to the reference level or concentration of said at least one biomarker indicates a need to administer to the subject a therapeutic treatment for kidney injury or CKD. In some embodiments, the biological sample is a urine or serum sample.

In another embodiment of this aspect, the method comprises (a) contacting biological sample obtained from a subject with at least one agent specific for at least one biomarker in a panel of biomarkers in Tables 1, 2, and 3; (b) measuring the level or concentration of the at least one biomarker using an assay specific for the at least one agent; and (c) comparing the level or concentration of the at least one biomarker with a reference level or concentration of the at least one biomarker, wherein an increase or reduction in the level or concentration of at least one biomarker in a panel of biomarkers in Tables 1, 2, and 3 in the sample relative to the reference level or concentration of said at least one biomarker indicates a need to administer to the subject a therapeutic treatment for kidney injury or CKD. In some embodiments, the biological sample is a urine or serum sample. The reference level contemplated herein may be based on a prior baseline test level of the same patient, or may be based on gathered test data for healthy patients without kidney injury and CKD, or both.

In another embodiment of this aspect, a method for monitoring treatment efficacy of a subject with kidney injury or CKD is provided, the method comprising: (a) determining, from a biological sample obtained from a subject at a first time point, a level or concentration of at least one biomarker in a panel of biomarkers in Tables 1, 2, and 3; (b) determining a level or concentration of said at least one biomarker in a panel of biomarkers from a sample obtained from said subject at a second time point; and (c) comparing the level or concentration of the at least one biomarker in a panel of biomarkers at the second time point to the level or concentration of the at least one biomarker in a panel of biomarkers at the first time point, wherein changes in the level or concentration of the at least one biomarker at said second time point indicates if the treatment is efficacious or not efficacious for said subject based on the component tested and if its level is trending in a direction indicative of improving CKD or lack of improving CKD.

### Systems for Determining Kidney Injury and CKD Status Based on Biomarker Levels and Concentrations

Other aspects of the invention also provide for systems (and computer readable media for causing computer systems) to perform a method for determining the expression value of kidney injury/CKD biomarkers.

In some aspects, embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

In one embodiment, a dedicated webpage in World Wide Web browser or a software along with specific algorithms utilizing machine learning and artificial intelligence can be used to perform analysis on test results of the kidney injury/CKD biomarkers for a given patient to assess status of kidney injury or CKD, also taking existing patient record and history into account.

Systems described herein are merely illustrative embodiments of the invention for performing methods of assessing whether an individual has kidney injury or CKD or is predicted to develop CKD, and are not intended to limit the scope of the invention. Variations of the systems described herein are possible and are intended to fall within the scope of the invention.

### Targeting or Utilizing the Kidney Injury/CKD Biomarker Proteins for Treatment of Kidney Injury and CKD

Other aspects of the invention provide methods for providing treatments for kidney injury or CKD, by targeting or utilizing the kidney injury/CKD biomarker proteins.

Accordingly, in one embodiment of this aspect, the method comprises (a) targeting the kidney injury/CKD biomarker protein (in Tables 1, 2, and 3) increased in kidney injury or CKD patient by antibody or drug to decrease the level of active said protein in the patient; (b) administering drug to increase the level of kidney injury/CKD biomarker protein (in Tables 1, 2, and 3) decreased in kidney injury or CKD patient; (c) administering peptides of the kidney injury/CKD biomarker protein (in Tables 1, 2, and 3) decreased in kidney injury or CKD patient to elevate the supply of the said protein in the patient; (d) administering peptides to reduce the activity of kidney injury/CKD biomarker protein (in Tables 1, 2, and 3) increased in kidney injury or CKD patient. Peptides used can have either endogenous sequences or modified sequences to maximize the effectiveness. Antibodies and drugs used can be obtained from either commercial source or developed by well-known methods.

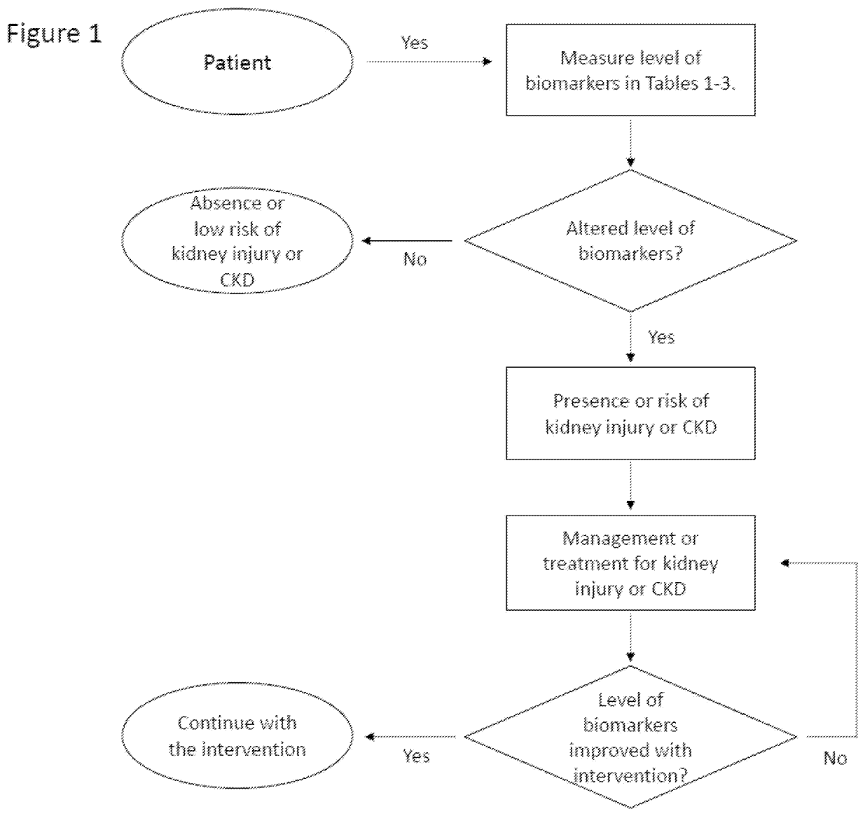

FIG. 1 shows an exemplary process for utilizing kidney injury/CKD biomarkers for detection and diagnosis of presence or risk of kidney injury or CKD in a patient and also for implementing a management or treatment intervention for a patient with presence or risk of kidney injury or CKD. Initially, a patient is identified and levels of one or more of the biomarkers listed in Tables 1-3 may be measured. If the measured kidney injury/CKD biomarkers are altered in a direction indicating presence or risk of kidney injury or CKD, an intervention such as management or treatment application may be implemented. If the kidney injury/CKD biomarkers are not altered, or not altered in a way to indicate presence or risk of kidney injury or CKD, then it can suggest the absence or a low risk of kidney injury or CKD. As noted throughout, measured levels may be compared to established baselines, or may be compared to prior recorded levels from the same patient. After a period of time under management or treatment, the levels of the kidney injury/CKD biomarkers may be measured again to determine if the levels are improving (i.e. moving toward a desired level) based on this intervention. If not, a different management or treatment approach may be considered. If yes, the intervention is continued and can be determined to be effective.

In one embodiment, the present disclosure may involve a method to determine if the patient requires implementation of a plan for preventing progression of kidney injury or CKD, comprising measuring the amount of selected biomarkers in a sample obtained from a subject, wherein the biomarkers are those listed in Tables 1, 2, and 3 to measure and determine whether the subject has any altered biomarker level indicative of potential progression in kidney injury or CKD. Examples of the sample may include, but are not limited to urine, blood, plasma, or serum. In some embodiments, the amount of the biomarker in the sample is measured by binding of an antibody or aptamer specific for the biomarker. In additional embodiments, the level or concentration of the biomarker protein is measured by measuring an activity of the biomarker.

In another embodiment, the present disclosure may involve a method for determining the efficacy of a treatment for kidney injury or CKD, comprising measuring the amount of selected biomarkers in a sample obtained from a subject receiving such treatment, wherein the biomarkers are those listed in Tables 1, 2, and 3 and comparing the level of the biomarkers to that of a sample from an untreated control to determine whether the treatment has had the effect of altering the biomarker level, or a reduction or maintenance in the stage of kidney injury or CKD or a slowing of progression through stages of kidney injury or CKD following treatment. Examples of the sample may include, but are not limited to urine, blood, plasma, or serum. In some embodiments, the amount of the biomarker in the sample is measured by binding of an antibody or aptamer specific for the biomarker. In additional embodiments, the level or concentration of the biomarker protein is measured by measuring an activity of the biomarker.

In further embodiments, the present disclosure may involve a method to utilize at least one kidney injury/CKD biomarker proteins in Tables 1, 2, and 3 as a target of therapeutic treatment (e.g., antibody or drug) or as a therapeutic agent (e.g., therapeutic peptide/protein) in kidney injury or CKD patients. In some cases, the target of treatment (e.g., antibody or drug) from kidney injury/CKD biomarker proteins in Tables 1, 2, and 3 in treatment of kidney injury or CKD patient is determined by using the methods disclosed herein and identifying at least one biomarker whose level is significantly altered in kidney injury or CKD patient.

In further embodiments, the present disclosure may involve A method of claim 17, wherein therapeutic agent (e.g., peptide/protein) derived from kidney injury/CKD biomarker proteins in Tables 1, 2, and 3 to be used in treatment of CKD patient is determined by using the methods disclosed herein, and identifying at least one biomarker whose level is significantly altered in kidney injury or CKD patient.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

1. A method for diagnosing kidney injury or chronic kidney disease (CKD) in a subject comprising the steps of: measuring a level or concentration of a normalizing agent (normalizing protein or normalizing factor) and measuring a level or concentration of at least one of the biomarkers in Tables 1, 2, and 3 in a biological sample obtained from a subject; and comparing the level or concentration of said biomarker with the level or concentration of a normalizing protein or factor, wherein an altered level or concentration of at least one biomarker indicates the presence of or risk of kidney injury or CKD.

2. The method of claim 1, wherein the concentration of the at least one biomarker protein is detected using an antibody-based or aptamer-based binding agent which specifically binds to the biomarker protein.

3. The method of claim 1, wherein the test values are analyzed through a specific algorithm to evaluate kidney injury or CKD status for a given patient for prevention or treatment plans.

4. The method of claim 1, wherein the level or concentration of the biomarker agent is measured by measuring an activity of the biomarker.

5. The method of claim 1, wherein the normalizing agent is creatinine, albumin, total protein, housekeeping gene or SDMA.

6. The method of claim 1, wherein the biological sample is a urine, serum, or plasma sample.

7. The method of claim 1, wherein the subject includes animals such as cat and dog and also human.

8. A method for diagnosing kidney injury or CKD in a subject in need thereof comprising the steps of: (i) measuring a level or concentration of a normalizing agent (normalizing protein or normalizing factor) in a biological sample obtained from a subject in need thereof; (ii) measuring a level or concentration of at least one of the biomarkers in Tables 1, 2, and 3 in the biological sample; wherein one or more agents are exposed to said biological sample prior to at least one of the step of said measuring of the level or concentration of the normalizing agent and said measuring of the level or concentration of said at least one biomarker; and (iii) comparing the level or concentration of said at least one biomarker with the level or concentration of the normalizing agent, wherein an altered level or concentration of at least one biomarker indicates the presence of or risk of kidney injury or CKD.

9. The method of claim 8, wherein the biological sample is a urine, serum, or plasma sample.

10. The method of claim 8, wherein the normalizing agent is creatinine, albumin, total protein, housekeeping gene or SDMA.

11. A method for diagnosing kidney injury or CKD in a subject in need thereof, the method comprising: (i) contacting a biological sample obtained from a subject in need thereof with at least one detectable agent specific for at least one of the biomarkers in Tables 1, 2, and 3; wherein one or more agents are exposed to said biological sample prior to at least one of the step of said measuring of a level or concentration of normalizing agent and measuring a level or concentration of said at least one biomarker; and (ii) comparing the level or concentration of said at least one biomarker with the level or concentration of normalizing agent, wherein an altered level or concentration of at least one biomarker indicates the presence of or risk of kidney injury or CKD.

12. The method of claim 11, wherein the biological sample is a urine, serum, or plasma sample.

13. The method of claim 11, wherein the normalizing agent is creatinine, albumin, total protein, housekeeping gene or SDMA.
